(19) United States
(12) Patent Application Publication
(10) Pub. No.: US 2009/0012372 A1
(43) Pub. Date: Jan. 8, 2009

(54) EXTERNAL SENSING FOR IMPLANT RUPTURE

(75) Inventors: Daniel Burnett, San Francisco, CA (US); Noel Johnson, Saratoga, CA (US); Gregory Hall, Redwood City, CA (US); Joseph Gryskiewicz, Edina, MN (US); Takashi Yogi, Santa Cruz, CA (US)

Correspondence Address: Mitchell P. Brook;LUCE, FORWARD, HAMILTON & SCRIPPS LLP, 11988 EL CAMINO REAL, SUITE 200, SAN DIEGO, CA, 92130, US

(73) Assignee: Novalert, Inc., Saratoga, CA (US)

(21) Appl. No.: 11/929,263

(22) Filed: Oct. 30, 2007

Related U.S. Application Data
(63) Continuation-in-part of application No. PCT/US2006/022761, filed on Jun. 12, 2006.
(60) Provisional application No. 60/855,247, filed on Oct. 31, 2006.

(51) Int. Cl. A61B 5/00; G08B 23/00

(52) U.S. Cl. 600/300; 340/573.1

(57) ABSTRACT

The present invention relates to a system and a method for sensing for the rupture of an implant (such as a breast implant) that has been implanted in body tissues or in an organ of a patient. In one embodiment, a system according to the present invention includes, among other possible things, a sensor coupled to an outer surface of the implant and configured to measure a property at the outer surface of the implant, for example, electrical conduction, chemical composition, or an optical property that is indicative of whether an implant rupture has occurred. The sensor is also configured to transmit a wireless signal to a device external to the body, which alerts the patient or a healthcare provider whether the measured property indicates that the implant rupture may have occurred.

### CROSS-REFERENCES TO RELATED APPLICATIONS

The present application claims priority to international application no. PCT/US2006/022761 filed on Jun. 12, 2006, which claims priority to provisional patent applications Nos. 60/688,882 filed on Jun. 10, 2005, 60/738,317 filed on Nov. 21, 2005, and 60/764,673 filed on Feb. 3, 2006, the entireties of which are incorporated herein by reference.

The present application also claims priority to provisional patent application Ser. No. 60/855,247 filed on Oct. 31, 2006, the entirety of which is incorporated herein by reference.

### FIELD OF THE INVENTION

The present invention relates to the field of medical devices. More particularly, the present invention relates to a system and a method for sensing for the rupture of an implant (such as a breast implant) that has been implanted in body tissues or in an organ of a patient.

### BACKGROUND OF THE INVENTION

An implant is a medical device that is introduced into the body of a patient through a surgical procedure. For example, a breast implant is a medical device that is surgically introduced either under breast tissue or under the chest muscle for breast augmentation or reconstruction and that is filled with a saline solution or with a silicone gel.

The primary components of a breast implant are the shell (also known as the envelope or the lumen), the filler, and a patch that covers a manufacturing hole. Different types of breast implants are known in the art to have different types of shell designs, fillers and constructive structures. For example, breast implants are currently available that exhibit a variety of shapes, profiles, volumes, areas, surface textures and thickness. Implant fillers are also currently available that are produced from silicone gels having the general composition of silicone oils, cured polymeric large silicones, and small amounts of uncured large and smaller silicones with minute amounts of metals, including a metal catalysts.

Breast implants typically have a limited life. A patient having breast implants may require additional surgeries during her lifetime due to rupture, other complications (for example, capsular contracture or breast pain), or unacceptable cosmetic outcomes (for example, asymmetry, unsatisfactory style and size, or wrinkling and rippling). More particularly, breast implants may rupture as a consequence of damage occurring during implantation or other surgical procedures, due to folding or wrinkling of the implant shell, due to trauma or other excessive force to the chest, or due to compression of the breast during mammography.

In 2001, the FDA published a study on the health effects of ruptured silicone gel breast implants, which was conducted out of concerns about the frequency and results of failures, ruptures and breakages (hereinafter collectively “ruptures”). Rupture is considered a concern because rupture of a silicone gel-filled implant may allow silicone to migrate through the tissues and because the relationship between free silicone and development or progression of disease is unknown. Moreover, implant rupture constitutes a device failure, in that the implant is no longer performing as intended, which in itself is believed to warrant an investigation.

The FDA study demonstrated that women with breast implant rupture diagnosed through magnetic resonance imaging (MRI) were no more likely than women with intact implants to report either persistent symptoms or doctor-diagnosed illnesses that were listed. Moreover, women with MRI-diagnosed extracapsular silicone gel (that is, silicone that had migrated outside the fibrous scar around the implant) were 2.8 times more likely to report the soft tissue syndrome known as fibromyalgia, which is characterized by widespread pain, fatigue, and sleep disturbance. Women with MRI-diagnosed extracapsular silicone gel were also found to be 2.7 times more likely to report that they had “other connective tissue disease,” a category that included a diverse group of illnesses such as dermatomyositis, polymositis, and mixed connective tissue disease.

Federal health advisers have recently recommended that silicone gel breast implants be allowed to return to the U.S. market after a 13-year ban and under strict conditions that will limit access. The FDA's advisers stated that one manufacturer of silicone-gel breast implants had performed more convincing research that indicated that only 1.4% of the implants rupture during the first three years after implantation, and had provided some evidence showing that breast implants may last as long as ten years. The FDA has stressed that sales should resume only if a manufacturer meets certain strict conditions, including: (1) that prospective patients sign consent forms that acknowledge implant risks, including that risk that the implant ultimately may break and/or require removal and/or replacement; (2) that silicone implants are sold only to board-certified plastic surgeons who complete special training to insert implants in a way that minimizes the likelihood of breakage; (3) that data about patients receiving implants be maintained in a registry to track the patients' long-term health; and (4) that formal studies be conducted to ascertain more definitively how often implants fail within ten years.

Today, silicone filled implants remain the implants of choice for many patients due to their superior look and feel. Prior to the FDA moratorium on silicone filled breast implants in 1992, the U.S. market consisted of 95% silicone-filled and 5% saline-filled breast implants. Concerns continue to persist in the medical community about chronic exposure to silicone gel and, in particular, about possible migration of silicone gel in the event of implant rupture. A large study supported by the National Cancer Institute (NCI) determined that most women with silicone gel implants will have a leak in their implants within ten years, which is unlikely to be detected unless the patient receives a MRI. While the NCI study was performed using the previous generation silicone-gel breast implant which was more prone to leakage, the current generation implants are also expected to have double-digit rupture percentages over the first ten years of use.

As breast implant ruptures typically do not cause immediate symptoms, implant patients are recommended to receive, at minimum, a MRI scan five years after the implant and then every two years thereafter and to have broken implants removed to minimize risk of silicone oozing into the breast or beyond. While MRI can be a useful tool for the detection of leakage, there are no signals or symptoms that indicate evaluation or monitoring of a breast implant should be performed.

The difficulty in detecting these ruptures, though, is highlighted by a recent meta-analysis of published studies, which found that the summary-sensitivity and specificity of MRI in breast implant rupture detection were 78% (95% confidence interval, 71-83) and 91% (95% confidence interval, 86-94), respectively. Therefore, even with MRI, the current platinum-iridium-standard for implant rupture detection, about 20% of all ruptures are likely to remain undetected and leave the patient at risk for chronic exposure to the silicone filler. Furthermore, almost 10% of patients will be subjected to unnecessary surgery for implant removal due to false rupture reports.

There are numerous other implantable devices that are currently used or being evaluated in medical practice. One such implantable device is an intragastric balloon that operates as a non-surgical, non-pharmaceutical alternative for the treatment of obesity and that is designed to induce temporary weight loss in an obese patient by partially filling the stomach so to help the patient achieve a feeling of fullness and adopt new dietary habits. This intragastric balloon is placed within the stomach endoscopically and is inflated with saline. Although the balloon can be deflated and removed endoscopically, it may improperly deflate during the course of therapy, leading to migration of the implant into the intestine with possible small bowel obstruction and subsequent surgery and even death.

Non-inflatable implants also are susceptible to loss of integrity following implantation. Given enough time, even titanium shells permit passage of bodily fluids. In fact, recalls for pacemakers, ICDS, and other implants commonly occur due to invasion of the implant by bodily fluids and subsequent malfunction, with sometimes life-threatening consequences.

Different attempts have been made in the prior art to improve implant safety. For example, U.S. Pat. No. 4,795,463 to Gerow discloses a prosthesis for implantation into human soft tissue that is constructed of a suitable implantable envelope and contents such as silicone gel, saline, or a combination of silicone gel and saline, to form a breast shape when implanted. The envelope is labeled with a marker that absorbs electromagnetic energy to an extent different from that of the envelope, its contents, and the human soft tissue in the breast cavity. This marker makes possible the use of roentgenographic imaging to determine whether the envelope has ruptured or whether the envelope is folded persistently in a particular location, thereby increasing the probability that the envelope may rupture along such a fold line. Also disclosed are a method for using roentgenography to determine whether contents have escaped from the envelope of the prosthesis by labeling the envelope with radioopaque materials, and a method for determining whether fold-fault rupture of the envelope of the implanted prosthesis is likely to occur.

U.S. Pat. No. 5,423,334 to Jordan discloses a system for enabling the acquisition from outside the body of a patient of data pertaining to a medical device implanted therein. A characterization tag is secured to the medical device prior to implantation, which is powered by energy absorbed through the mutual inductive coupling of circuitry in the characterization tag with an alternating magnetic field generated outside the body of the patient. That circuitry in the characterization tag is selectively loaded and unloaded in a predetermined sequence of loading conditions that correspond to data about the implanted medical device. The alternating magnetic field is generated in a characterization probe, which is moveable external to the body of the patient and which includes electrical circuitry for sensing variations in the amount of energy absorbed from the field by the characterization tag. The characterization tag is secured to the exterior of the medical device by a biocompatible potting material in a characterization tag recess or, if the medical device is assembled from a plurality of constituent parts, by permanently capturing the characterization tag between a pair of these parts.

U.S. Pat. No. 5,496,367 to Fisher discloses a breast implant that includes an elastomeric envelope adapted to contain a fluid material and baffles inside the envelope. The baffles are provided to reduce or dampen wave or ripple action and motion of the fluid material contained by the envelope when implanted in a breast.

U.S. Pat. No. 5,833,603 to Kovacs et al. discloses a biosensing transponder for implantation in an organism that includes a biosensor for sensing one or more physical properties related to the organism after the device has been implanted, including optical, mechanical, chemical, and electrochemical properties, and a transponder for wirelessly transmitting data corresponding to the sensed parameter value to a remote reader. Disclosed embodiments utilize temperature sensors, strain sensors, pressure sensors, magnetic sensors, acceleration sensors, ionizing radiation sensors, acoustic wave sensors, chemical sensors including direct chemical sensors and dye based chemical sensors, and photosensors including imagers and integrated spectrophotometers. The transponder includes an energy coupler for wirelessly energizing the device with a remote energy source, and a control circuit for controlling and accessing the biosensor and for storing identifying data. The energy coupler can be an inductive circuit for coupling electromagnetic energy, a photoelectric transducer for coupling optical energy, or a piezoelectric transducer for coupling ultrasonic energy. The control circuit can be configured to delay, either randomly or by a fixed period of time, transmission of data indicative of the sensed parameter value to thereby prevent a data collision with an adjacent like device.

U.S. Pat. No. 6,755,861 to Nakao discloses a method of breast reconstruction that uses a breast prosthesis having a plurality of chambers or compartments distributed through a body member or shell in the form of a breast. The chambers are disposed along the superior, lateral, and inferior surfaces, as well as in the interior, of the body member. The chambers are differentially pressurized or filled, in order to control the shape of the prosthesis upon implantation thereof. Valves are provided for regulating the flow of fluid into and from the chambers, and the prosthesis and the fill levels of the respective chambers may be selected by computer. This implant provides for a plurality of one-way valves, each disposed between two adjacent chambers for enabling a transfer of fluid from one of the adjacent chambers to another of the adjacent chambers.

U.S. Patent Publication 2005/0033331 to Burnett et al. discloses a gastric balloon implantation device that may incorporate a visible dye or marker to enable detection of device rupture.

U.S. Patent Publication 2005/0267595 to Chen et al. discloses a gastric balloon implantation device which includes as a leak monitoring system, a sensor that comprises a fine lattice or continuous film of detection material embedded in the wall or in between layers of the wall covering the entire device.

U.S. Patent Publications 2006/0111632 and 2006/0111777, both to Chen, disclose various implantation devices including breast implants which include as a leak monitoring system a sensor that comprises a fine lattice or continuous film of detection material embedded in the wall or in between layers of the wall covering the entire device.

### SUMMARY OF THE INVENTION

Devices and methods are provided for external sensing for rupture of an implant, for example, of a breast implant filled with a silicone gel. These devices operate by causing a sensor to communicate with an external device alerting a user or a healthcare provider that the integrity of the implant is failing.

One embodiment of the present invention relates to a system for external sensing for implant rupture that includes, among other possible things, a sensor coupled to an outer surface of the implant and configured to measure a property at the outer surface of the implant, for example, electrical conduction, chemical composition, or an optical property that is indicative of whether an implant rupture has occurred. The sensor is also configured to transmit a wireless signal to a device external to the body.

The sensor may be provided as a separate component coupled to the outer surface of the implant or may be printed on the outer surface of the implant. Among possible locations where the sensor may be disposed on the outer surface of the implant, the sensor may be bonded or vulcanized to a patch closing a manufacturing hole in the implant, or may be lodged in a recess provided in a reinforced area of the outer surface.

In one embodiment, the sensor comprises a plurality of electrical leads coupled to the outer surface of the implant, and the sensor is structured to measure electrical conduction between those electrical leads. Preferably, the electrical leads are electrodes arranged on the outer surface of the implant to provide a profile flush with the outer surface, in particular, with the outer surface of the patch. The sensor may also include a multi-vibrator oscillator that has a frequency determined by a resistance between the electrical leads and that includes an astable multivibrator. The sensor may further include a microprocessor that detects a change in the measured property by comparing a reading of that property against a predetermined threshold.

Other embodiments of the present invention are configured to measure spectrophotometric, visual, pH, chemical, pressure, viscosity, distention or other properties indicative of whether an implant rupture has occurred.

The sensor may also include a radio-frequency identification circuit. If the implant has a filler with insulating properties such as silicone gel, the sensor measures a reduction in electrical conduction after the implant rupture, for example, due to a partial or total coating of the electrical leads by the implant filler.

The signal provided by the sensor to the wireless device may include data, for example, measurements of an electrical or other property, and, in one embodiment, may be transmitted to the external device at a frequency of about 13.56 MHz.

The sensor may also be configured to receive power transmitted from the device, so to activate the sensor and initiate the desired measurement, or may include an autonomous power source, for example, a battery that dispenses power upon interrogation of the sensor by the external device. When the external device provides power to the sensor, such power may be provided inductively with about one Watt of radio-frequency output.

The external device may be configured to be hand held and, in one embodiment, includes a coil that couples to a second coil in the sensor to provide power to the sensor inductively. The external device may be actuated by depressing a button that connects to the sensor and may display (for example, by lighting one or more light emitting diode or LED) whether the property measured by the sensor indicates that the implant is intact or that a rupture has occurred. Alternatively, the external device may emit an alert that provides a vibratory, acoustic, visual, tactile, or other stimulus.

According to the response generated by the external device, the patient or an attending healthcare provider receives a first indication of whether a follow-up examination of the patient with MRI equipment is advisable.

Methods of use of the systems described hereinbefore are also provided.

Another embodiment of the present invention relates to a system for external sensing for implant rupture that includes, among other possible things, a sensor configured to detect a rupture in the external shell of the breast implant, and a signaling element located in a lumen or on the shell or outside of the breast implant, wherein the signaling element is configured to be triggered by the sensor to alert the user or a healthcare provider of the rupture.

Still another embodiment of the present invention relates to a system, in which a thin electrical contact liner is coated on the skin of an implant and in which the conductive layer optionally has a larger surface area or volume than the lumen. This sensor may be triggered by any rupture in the integrity of the skin (or outer layer) of the implant, detected through changes in conductivity or other properties associated with the liner. The signal may be triggered by a breakage, stretching, or displacement of any of these wires.

In any of the foregoing embodiments, the shell of the implant may be flexible or rigid. The sensor may also include a mesh incorporated throughout the shell of the implant and may be configured to detect alterations in the external portion of the shell based on electrical, chemical or physical changes to the mesh.

In any of the foregoing embodiments, the external device may incorporate a second signaling element to alert the user that recharging is required. Further, the second signaling element may be a vibratory, acoustic, visual, tactile, electromagnetic field or other stimulus. Still further, the external device may communicate through ultrasound, radiofrequency or electromagnetic fields, and the receiver may also receive information that allows for programming, resetting or other manipulation of the system.

In any of the foregoing embodiments, the external power source may be located within or near a bed, couch, chair or seat of the user, or within accessories, clothing, personal items, house, car or workspace of the user. The power source may be battery and/or capacitor powered and may be rechargeable, for example, by connecting to a standard wall outlet. Additionally, the external powering may be continuous when the implant is within a predetermined range of the external power source or of an external signal transmitter, or the external powering and/or signaling may be intermittent with an at least weekly, monthly or yearly interaction with the implant.

In any of the foregoing embodiments, the implant may be radiolucent.

In summary, the systems and methods for external sensing for implant rupture according to the present invention provide relevant information on the integrity of body implants (for example, of silicone gel implants) by detecting the presence of a breach in the shell of the implant in a manner that is faster and more convenient than MRI.

As used herein, the term “shell” refers to the exterior portion of an implant device which functions to separate the interior contents from body tissue and fluids. In a preferred embodiment, the shell has a thickness of 0.0.05-5 mm and a durometer value of 20 A-90 A for hardness.

As used herein, the term “lumen” refers to a cavity that is present inside the shell of an implant.

These and other features, aspects, and advantages of the present invention will become more apparent from the following description, appended claims, and accompanying exemplary embodiments shown in the drawings.

### BRIEF DESCRIPTION OF THE DRAWINGS

The drawings constitute a part of this specification and include exemplary embodiments of the invention, which may be embodied in various forms. It is to be understood that in some instances various aspects of the invention may be shown exaggerated or enlarged to facilitate an understanding of the invention.

FIG. 1 illustrates a perspective view of an embodiment of the invention, in which a breast implant contains a sensor and a signaling/alerting element disposed in the lumen of the implant.

FIG. 2 illustrates a perspective view of an embodiment of the invention, in which a breast implant contains a mesh sensing a breach of the implant and coupled to a signaling element.

FIG. 3 illustrates a perspective view of an embodiment of the invention, in which a breast implant includes a plurality of sensors disposed in the shell of the implant and coupled to a power source and an external communication component.

FIG. 4 illustrates a perspective view of an embodiment of the invention, in which a breast implant includes sensing and communication elements connected to a communicating/recharging ring by a tether that also channels fluid from the inside of the implant shell to the sensing element.

FIG. 5 illustrates a side view of an embodiment of the invention adapted for an implant with a rigid housing.

FIGS. 6A-6B illustrate the interaction of an implant (for example, a breast implant) with the system for external sensing depicted in FIG. 4.

FIGS. 7A-7C illustrate perspective views of the function of a system for external sensing of breast implant rupture, in which a sensor is powered and/or interrogated externally.

FIGS. 8A-8C illustrate an embodiment of the invention, in which a sensor is coupled to the patch of the implant. More particularly, FIG. 8A illustrates the basic configuration of the sensor disposed on the patch, FIG. 8B illustrates the configuration of the sensor-patch combination when the implant is intact, and FIG. 8C illustrates the configuration of the sensor-patch combination when a breach has occurred in the implant.

FIG. 9 illustrates a perspective view of an embodiment of the invention, in which a sensor is coupled to the patch of a breast implant and detects changes of conductivity on the milieu surrounding the implant.

FIG. 10 illustrates a perspective view of a second embodiment of the invention, in which a sensor is coupled to the patch of a breast implant and detects changes of conductivity on the milieu surrounding the implant, and in which additional sensors are disposed on the shell of the implant.

FIGS. 11, 11A and 11B illustrate perspective views of an embodiment of the invention, in which a sensor is coupled to the patch of a breast implant and detects changes of conductivity on the milieu surrounding the implant (FIG. 11) and further illustrate two possible configurations of the sensor (FIGS. 11A-11B).

FIGS. 12, 12A and 12B illustrate perspective views of an embodiment of the invention, in which a sensor is coupled to the patch of a breast implant and detects changes of conductivity on the milieu surrounding the implant. More particularly, FIG. 12 illustrates an embodiment in which additional sensors are disposed on the shell of the implant, and FIGS. 12A-12B illustrate two possible configurations of those sensors.

FIG. 13 illustrates a top view of an embodiment of a sensor as usable in the preceding figures, and further illustrates relative size of the depicted sensor with a U.S. ten cent coin.

FIG. 14 is a diagram illustration of a circuit used in the sensor depicted in FIG. 13.

FIG. 15 illustrates a perspective view of an emitter and receiver wand for transmitting energy to a sensor coupled with an implant and reading a signal or data received from the sensor.

### DETAILED DESCRIPTION OF EMBODIMENTS OF THE INVENTION

Detailed descriptions of embodiments of the invention are provided herein. It is to be understood, however, that the present invention may be embodied in various forms. Therefore, the specific details disclosed herein are not to be interpreted as limiting, but rather as a representative basis for teaching one skilled in the art how to employ the present invention in virtually any detailed system, structure, or manner.

The present invention provides systems and methods for monitoring leakage from or into a bodily implant by sensing and communicating the occurrence of loss of integrity in the shell of the implant. More particularly, an implant monitoring system constructed according to the principles of the present invention includes a sensor coupled to the shell of the implant and a signaling element for external communication.

The system of the present invention may include an internal power source and may employ software to allow for programmability and/or interrogation of the device, or may be recharged and/or powered through an external source. Circuitry associated with the sensor, the signaling element, external powering, and/or external interrogation may be composed of resistors and capacitors and, in certain embodiments, may be printed onto a patch of the implant. The wireless communication to external devices may be done using RFID circuitry that, in some embodiments, may be coupled to the patch or printed on the patch.

The sensor is designed to detect changes in different properties. For example, the sensor may detect changes in conductivity, wall pressure, fluid pressure, pH, salinity, hydration, electrical fields, etc., and may also detect the presence of specific markers found in surrounding body tissues or in other potential markers that are indicative of rupture.

An embodiment of the sensor includes one or more thin electrical contact liners embedded in the shell of an implant. This sensor may be triggered by any rupture in the integrity of the shell, which may be detected through changes in conductivity or other properties. An alternative embodiment of the sensor may involve thin filament wires (or fibers of any type) placed in a meshwork throughout the shell of an implant, which trigger a signal upon a breakage, stretching, or displacement of any of these wires.

Another embodiment of the sensor includes a switch that is triggered once certain conditions are met, thus preserving the power within the implantable power source for signal generation. For example, two leads may be positioned within a space that contains a polymer that degrades in the presence of silicone gel or other predetermined material. The polymer forms a barrier that separates the two leads and, once introduced to the silicone gel of a ruptured implant or to another pre-determined material, the barrier rapidly degrades, triggering the sensor.

In another embodiment, two leads may be positioned within a space that contains a desiccated hydrophilic polymer, which may be coated by an aqueous barrier that dissolves on the presence of bodily fluids (for example, ions, proteins, glucose, etc.). Once introduce to bodily fluids, the aqueous barrier rapidly degrades, exposing the hydrophilic polymer to water and causing the hydrophilic polymer to expand. Such an expansion acts to connect the two electrodes and complete a circuit that causes the activation of the signaling element.

The introduction of an aqueous barrier may be particularly advantageous when it is to be expected that the implant may be penetrated by water vapor. For example, silicone-encased implants may be exposed to large amounts of water vapor upon a rupture of the implant so that, once the silicone shell ruptures and the aqueous barrier is rapidly compromised by bodily fluids, a hydrophilic polymer rapidly swells and closes the circuit to generate the rupture alert. In the absence of such an aqueous barrier, there may be a large number of false positives due to water vapor causing expansion of the hydrophilic polymer. In contrast, in instances in which the shell of the implant is relatively impermeable to water and other vapors, the aqueous barrier coating may be unnecessary, enabling the use of a hydrophilic polymer (or other bodily fluid sensor). This is but one embodiment of the present invention. Other embodiments exist in which the switch may be triggered by changes in salinity, pressure, pH, hydration, or other components found external to the implant.

Once the sensor conditions have been met, the alert mechanism for the device delivers a device rupture signal to the patient and/or to a healthcare professional. The alert mechanism may communicate the occurrence of integrity failure for an implant via a plurality of different patient-centered stimuli, including a visual stimulus (for example, activation of a light visible through the skin), a palpatory stimulus (for example, vibration) or an auditory stimulus (for example, emitting a beeping sound). The vibratory alert signal, for example, could be either constant or intermittent in nature and would be intended to be forceful enough not to be mistaken for other body sensations. This alert may be programmed to be sensed solely by the patient (for privacy concerns) and not to interfere with sleep, but, at the same time, not to be easily ignored. The triggering of this alert mechanism would signal to the patient and/or a healthcare professional that the device needs to be inspected.

Alternatively, the device may communicate the existence of a rupture to an external device via radio-frequency or electromagnetic fields. In those instances in which the power source is internal and is rechargeable, these signaling mechanisms may also be triggered to inform the patient or the healthcare provider that the device requires recharging.

The signaling element provides for an exchange of information with an external device. In one embodiment, the device may contain internal programming capabilities that allow for monitoring of changes in the implant that are indicative of a rupture of the device and at the same time adjust a baseline for monitoring these changes. This feature may be used as a safeguard to ensure that the patient is not subjected to unnecessary surgeries prompted by false positives in instances in which the device could have been safely reprogrammed externally.

A preferred embodiment of the present invention relates to a system for externally sensing the rupture of a silicone gel-filled breast implant by monitoring a condition on the outer surface of the implant. In the event that the outer silicone envelope of a silicone gel-filled breast implant fails, silicone gel may exit the implant and come into contact with one or more sensors disposed on the outer surface of the implant. The sensor may be structured as two or more electrodes that are electrically connected and that are frequently interrogated for conductivity. When the silicone gel coats one or more of the contacts, conduction between the contact decreases or is terminated, opening the circuit between the electrodes. An external reader can then detect this drop or termination of conduction between the electrodes, providing an alert that the breast implant may have been ruptured. This embodiment will be described in a greater detail in a later part of this paper.

Thus, by having a simple receiver/transmitter in the breast implant powered by the external reader (for example, an RFID chip on the patch of the implant), a system constructed according to the principles of the present invention will have a minimal impact on the profile of the breast implant, be unlikely to be felt upon breast palpation, and function for the life of the implant. In this embodiment, the patient will simply have to ensure that she comes in contact with the power/signal transmitter, which could be placed in the patient's home (for example, at her bedside for daily or more frequent checks) or in a physician's office (for less frequent checks). The power/signal transmitter could then contact the physician or healthcare provider automatically and/or alert the patient and/or alert an examining healthcare provider. In the event that the patient is alerted, the previously trained and educated patient would then contact a healthcare professional to have her device interrogated (that is, to have a MRI or other appropriate investigation initiated) and/or to have surgery to remove the implant. As a result, a patient would know relatively immediately about rupture of an implant and would not have to wait, in some cases up to five years or more, to have a regularly scheduled MRI. Alternatively, a healthcare provider may examine a patient having breast implants for leaks during regularly scheduled check-ups. In other embodiments, the system of the present invention may incorporate a battery that could be rechargeable in nature or may incorporate a battery that has a life-time functional expectancy (i.e., having a very-low-current-draw sensor or a zero-current-draw switch activated device).

The device of this application may be used in conjunction with any implantable technology. Although breast implants are specifically mentioned as examples, the nature of this device makes it applicable to all forms of implants, including implantable gastric balloons. In such gastric embodiments, as with the breast implant embodiments, one scenario involves the use of an external power/signal generator that communicates with a receiver/transmitter (for example, a RFID chip) associated with the shell of the gastric implant. The RFID chip may be located within the shell, printed on the outside of the shell, attached to the inside wall of the shell, located in the lumen of the shell but not attached to the shell wall, or coupled to the outer wall of the shell. These options also are applicable to other types of implants.

The gastric balloon may be inflated with a solution that is non-conductive, or that is less conductive than normal saline, but is osmotically active. Thus, upon ingress of bodily fluids into the failing implant, as in the case of the breast implant, the conductivity across the electrodes within the implant or printed on the inside of the shell of the implant will be altered, and this information will be transmitted externally via the RFID mechanism coupled to the electrodes. This mechanism has been validated by the inventors in a protocol that found that the capacitance of a solution of deionized water is on the order of picofarads across electrodes spaced five millimeters apart, while normal saline capacitance across this gap is on the order of 10 to 100 nanofarads (a 1000-fold difference). The relationship is nearly linear such that even a small amount of saline or gastric fluid is capable of registering a significant difference in capacitance or conductivity, which may be transmitted via the coupled RFID electronics. Thus, by filling the implant with psyllium fiber (or another osmotically active, FDA-cleared substance that is either more or less conductive than normal saline or gastric secretions), the conductivity, capacitance, resistance, etc. across the electrodes within the implant may be checked intermittently or continuously. Further, if a change in any of these parameters is found, the device may be rapidly replaced prior to dangerous passage into the intestine.

If the implanted device is inflated at the time of the surgical procedure, a fluid with a conductivity, resistance, or capacitance which deviates from that of normal saline or bodily secretions may be employed, in order to use electrodes to measure the change in electrical parameters that are indicative of implant rupture. The filling fluid may also be significantly different with respect to the chemical, optical, physical, pH, and/or electrical properties of normal saline and/or the fluid surrounding the implant, such that these parameters may be sensed within the implant as well. Changes to any one of these, or other, parameters within the implant may indicate rupture of the external implant barrier.

The present invention may also use scaffolding and/or other support structures in combination with the aforementioned rupture sensing technologies. Some of these scaffolding and support structures are disclosed in U.S. Patent Publication 2005/0033331, which is incorporated herein by reference in its entirety. These support structures may ensure that the device does not deflate and cause problems (for example, intestinal obstruction in the case of the gastric balloon) in the event of a catastrophic rupture and/or rapid leak. Such a support structure may also be easily engaged and collapsed with standard endoscopic tools, such as endoscopic snares, forceps or scissors, thereby providing a significant advance over the current removal procedures of a gastric device. One advantage of this embodiment is that the device may be extracted from the stomach of the patient without the need for cumbersome and unwieldy puncturing, which is typically necessary with current gastric balloon removal.

Some of the advantages of an external sensing system, according to the present invention, include a continuous (or intermittent but frequent) monitoring of implant integrity, an implant rupture signaling mechanism for both the patient and healthcare professional, and the ability to have a sensor communicate with an external device information about the state of an implant. In particular, these benefits may be obtained in the preferred embodiment by modifying only the patch of the implant, which is the most durable, tear-resistant portion of the implant.

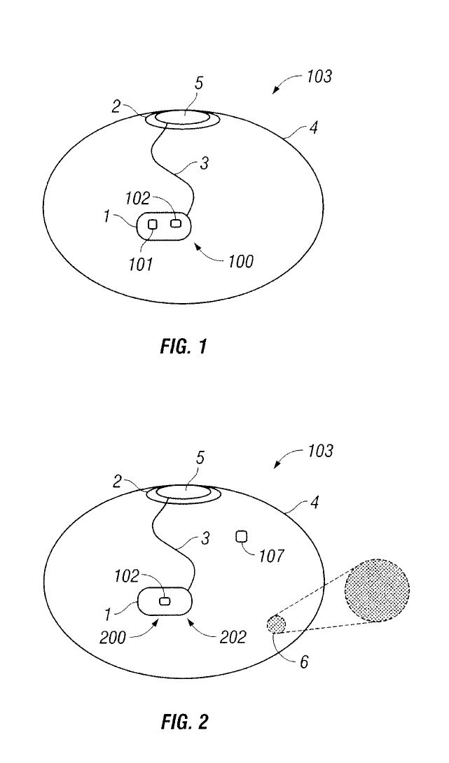

Some of the embodiments disclosed hereinabove will now be described in greater detail. Referring first to FIG. 1, a first embodiment of an external sensing system 100 for a fluid- or gas-filled implant 103 according to the present invention includes a sensor 101, a signaling or alerting element 102, and other electronics (not labeled) that are incorporated into an internal element 1 that is housed within an open space or cavity defined by an exterior shell 4 of implant 103. As a result, sensor 101 of this embodiment is not provided as a continuous film or as a mesh on shell 4.

Shell 4 may include an injection/inflation patch 5, which is designed to plug an inflation opening and which generally defines a discrete region of increased durometer and/or thickness through which implant 103 may be inflated or filled.

As shown, device 100 may also include an optional communicating/inductive charging ring 2 and a connecting tether 3 that connects charging ring 2 to internal element 1. In turn, internal element 1 senses and communicates externally if there has been a rupture of shell 4. In response to a signal received from sensor 101, signaling element 102 within internal element 1 may vibrate, communicate to an external device (not shown), make an audible noise, or emit a light to indicate that a check is required to ensure integrity of shell 4. Signaling element 102 may also alert the user that recharging of her device 100 is required in those embodiments in which device 100 is internally powered. In both the internally and externally powered embodiments, device 100 may communicate externally and be programmable/resettable such that if it is triggered without a rupture of the shell 4, it can simply be reset to continue monitoring.

Although system 100 is shown monitoring an implant 103 having a spherical shell 4 (as would be the case for many breast implants and gastric balloons), this is but one embodiment of device 100, and other embodiments contemplate non-spherical shapes. Moreover, device 100 may be adapted to monitor implants in any area of the body and may be made of any material.

Sensor 101 within internal element 1 may be one or more of a variety of sensors including sensors for detecting changes in salinity, pH, hydration, chemical markers (or other compounds), pressure, impedance, conductance, or other physical properties within the monitored device. Moreover, sensor 101 may use electric, spectrophotomoteric, chemical or physical measurement technologies.

Alternatively, device 100 may use a passive sensor that does not require active measurements of the internal milieu, but instead remains dormant until the appropriate conditions are met, in particular, until a rupture of implant 103 occurs. This type of sensor includes sensor containing pH- and/or ion-sensitive polymers, which may swell, degrade, or alter their physical properties in some manner that allows electrodes to come in contact with each other, thereby signaling a rupture of the implant 103. An embodiment of this design may involve the use of a pH-sensitive compound (for example, a pharmaceutical enteric coating) that is placed between the electrodes of the alerting element 102 and remains there until aqueous fluid enters the implant 103. At this point, the polymer degrades and the electrodes come into contact alerting the user of a rupture. Materials that are suitable for this application are Eudragit (Rohm and Haas) and Opadry AMB (Colorcon). The only requirement is that the sensor 101 be resistant to compounds normally found within the monitored device (for example, water vapor), but be triggered upon influx of abnormal materials (for example, ions or proteins).

Alerting element 102 within internal element 1 may be one or more of a variety of possible signal generating devices, including physical stimuli generators and/or energy or electromagnetic communicators. Among the possible physical stimuli are auditory (for example, a sound), visual (for example, a light under the skin) and tactile (for example, a vibration). In particular, vibration may be essentially soundless and satisfy both privacy concerns and the desire to communicate robustly. In the case of vibration, a small eccentric motor, piezoelectric element or very low-range acoustic element may be used to generate the intended vibration. Any source of vibration or energy-delivery could be used, though, with the only requirement being that the patient be sufficiently alerted.

The alert may be activated during certain time periods, over intervals, or with a unique signal to indicate device conditions. For example, in the case of a rechargeable device, if the device requires recharging, the alert may be of a certain nature so as to indicate that the battery is low, as opposed to a signal for implant rupture. Moreover, once alerted, the healthcare provider may, in one embodiment, be able to interrogate device 100 and even reprogram the sensitivity threshold when a sensor 101 with a slow baseline drift is employed.

In the case of a device without an internal battery, alerting element 102 and/or sensor 101 may be powered externally via inductive, RF or EMF energy generation to provide for intermittent, non-continuous interrogation of device 100. The interrogating device (not shown) may be an office-based device for routine checks or a home-use device designed to interrogate the device 100 automatically and to report (to the user or healthcare provider) that the implant 103 has failed. The interrogating device is placed in an area in which the patient can interact with it on a daily basis to allow for regular, but intermittent, interrogation of the device 100 with subsequent rapid reporting. This reporting could, again, be a local activity signaling the user, or could be directly transmitted to the healthcare provider to allow for immediate action.

FIG. 2 illustrates another embodiment of an implant integrity monitoring device 200. As can be seen in this embodiment, in contrast to the design of FIG. 1, sensor 202 is not part of internal element 1 but is instead incorporated into a mesh 6 of implant 103. Mesh 6 may be incorporated into shell 4, may be just inside shell 4, or may be just outside of the shell 4. Signaling element 102, circuitry and all electronics other than the optional communicating/inductive charging ring 2 and connecting/recharging tether 3 are still incorporated into an internal element 1. However, tether 3 may be used to transfer information between sensor 202 within mesh 6 and internal element 1 (which can actually be located anywhere within the shell and does not need to be centrally located). In this embodiment, alterations to external shell 4 can be detected by changes in volume, impedance, conductivity, magnetic field, etc. which may arise as a result of a break in the sensing mesh 6.

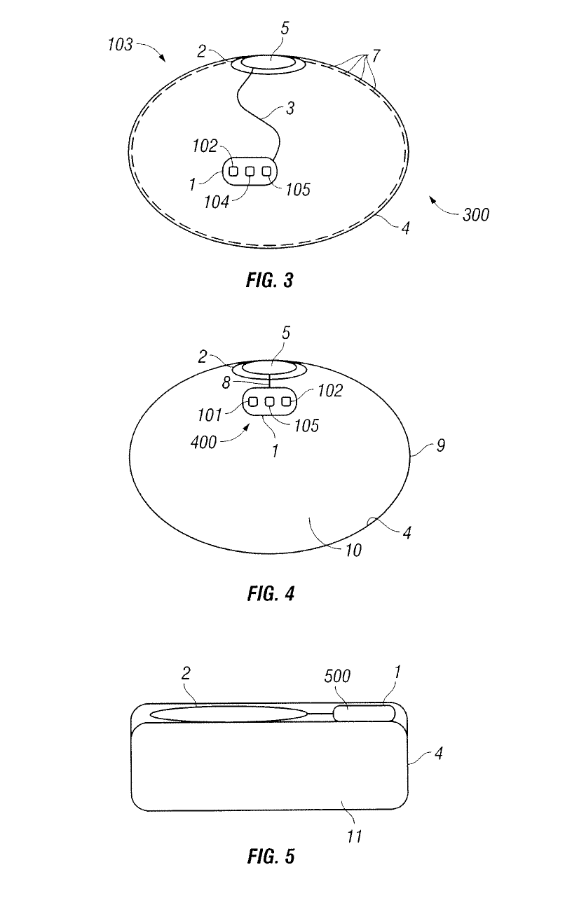

FIG. 3 illustrates another embodiment of an implant integrity monitoring device 300, in which sensors 7 are interspersed throughout a shell 4 of implant 103. Internal element 1 may contain a power source 104, an external communication component 105, and/or a signaling or alerting element 102. Moreover, internal element 1 may communicate with sensor 7 in external shell 4 via a communicating/recharging tether 3. Alternatively, and this goes for all embodiments, internal element 1 may be affixed to the internal surface of shell 4 of the implant 103 at one or more points requiring little, or even no, tether. Sensors 7 inside of, or within, shell 4 may detect influx of external components from tissue (for example, breast tissue) surrounding implant 103. For example, sensors 7 may be hydration sensors or salinity sensors. Alternatively, the sensors may be pH, conductivity, impedance, light, or chemically-based. There may also be multiple sensors 7 in regions of high-risk (for example the inflation patch and/or a manufacturing seam). Once again, this is but one embodiment of the present invention and it maybe adapted to monitor implants in any area of the body and may be made of any material.

FIG. 4 illustrates another embodiment of a system for external sensing for implant rupture 400 according to the present invention. Although sensing element 101, communicating element 105, and alerting element 102 may be separately provided throughout implant 103, they may, as shown, be incorporated into one internal element 1, which communicates with the optional communicating/recharging ring 2 via a recharging tether 8 that has additional properties. In this embodiment, tether 8 also channels fluid from the inside of shell 4 to sensing element 101 within internal element 1. Further, the inside of shell 4 may be coated with a coating material 9 designed to bring the sensed substance to sensing element 101 within internal element 1. Coating material 9 may be, for example, parylene or heparin hydromer and may be designed to carry the ionic bodily fluid to sensor element 101 at which ion- or pH-sensitive sensor element 101 may be triggered, thereby alerting the patient and/or healthcare provider of an implant rupture. This design will be particularly useful for indications in which the filling of implant 103 is relatively impervious to the substance being sensed. A good example is the silicone gel breast implant, which, when filled with silicone gel 10, discourages influx of any aqueous material. The internal coating material 9, though, allows the aqueous fluids to track around gel 10 to tether 8, from which the fluids may be carried to sensor element 101 within internal element 1. Coating material 9 may consist of any one or more of a variety of materials, including, as previously mentioned, parylene and/or heparin hydromer. These compounds may coat tracks within silicone shell 4 (in the breast implant configuration) or may coat the entire inside of shell 4. This will help in generating a potential space between gel 10 and shell 4 (in the case of parylene) and/or to attract aqueous fluid due to hydrophilicity (in the case of hydrophilic polymers such as the heparin hydromer coating). Whether drawing the fluid around to sensor element 101 or creating a plane for the fluid to track within, either mechanism could be used if the desired rate of fluid ingress is not found to occur spontaneously in an unmodified implant 103. In a variant of the present embodiment, the coating may direct a substance of interest (for example, a bodily fluid) towards electrical leads (for example, electrodes) that are included in sensing element 101, establishing or hindering electrical conduction between those electrical leads.

FIG. 5 illustrates another embodiment of a system for external sensing of implant rupture 500. In this embodiment, device 500 is incorporated into an internal element 1 that is provided adjacent to an external shell of an electronic device 11. As shown, device 500 is minimized to allow for incorporation into a small space from which the device may monitor electronic device 11, which could be, for example, a pacemaker, an implantable pump, an implantable glucose sensor, an implantable cardioverter defibrillator, an implantable left ventricular assist device, or any other implantable device with electrical components.

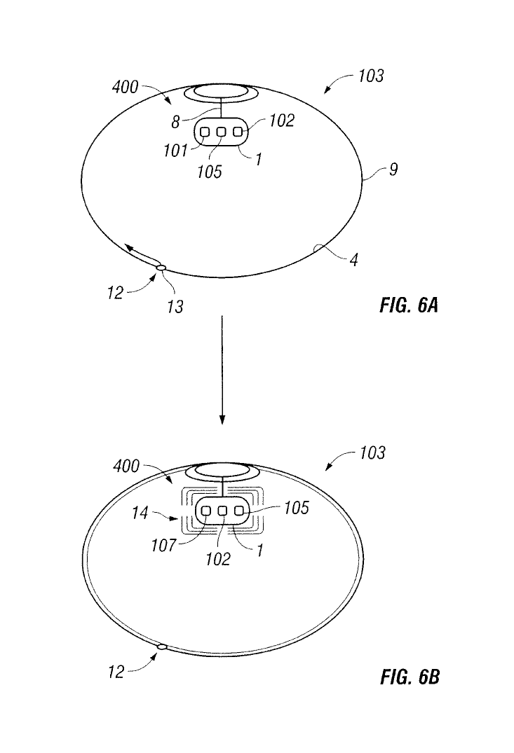

FIGS. 6A and 6B illustrate the interaction of an implant 103 (for example, a breast implant) with the system for external sensing 400 from FIG. 4. Implant 103 is shown with a rupture 12 in its shell 4. Rupture 12 allows fluid 13 to track around the inside of shell 4 along optional coating material 9 to sensor element 101 within internal element 1 via optional tether 8. Once fluid 13 has made it way from the site of rupture 12 to the inside of implant 103 and reaches internal element 1, sensor element 101 (which may be, for example, a pH or salinity sensor) is triggered due to its exposure to the constituents within bodily fluid 13. Once sensor element 101 is triggered, signaling or alerting element 102 (which maybe, for example, an eccentric motor) may, as shown by reference character 14 in FIG. 6B, vibrate rapidly. This is but one of several alerting mechanisms, with auditory signals, visual signals, and wireless communication being three other possibilities among many possible. These are exemplary illustrations, though, and should not be interpreted to be the only possible embodiments.

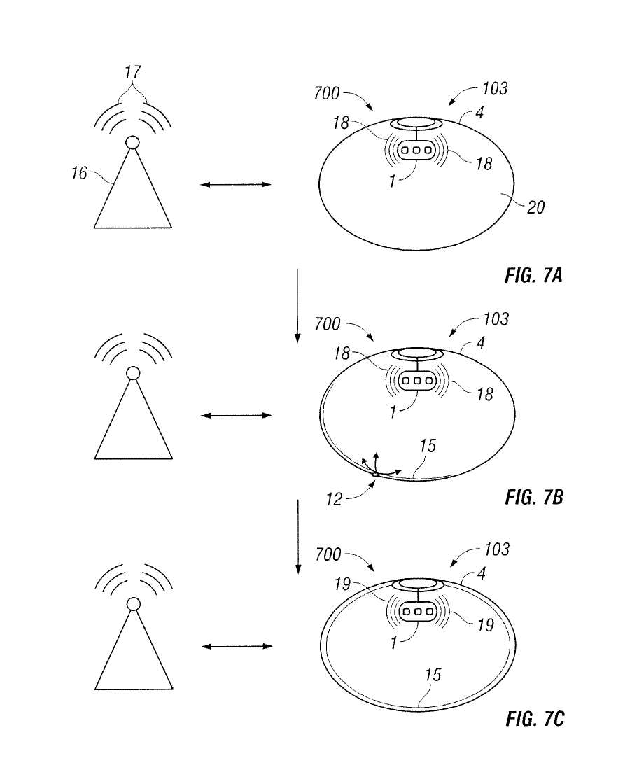

FIGS. 7A-7C illustrate perspective views of the function of a system for external sensing of implant rupture 700 for breast implants, in which implant 103 is powered and/or interrogated externally. In this embodiment, a power and/or signal emitter/receiver 16 emits a radiofrequency or electromagnetic waves 17 to power and/or communicate with implant 103. In turn, internal element 1 of device 700 emits a signal 18, 19 in response to the power and/or signal emitter/receiver 16. This signal 18, 19 may then be interpreted by power and/or signal emitter/receiver 16, thereby alerting the user and/or healthcare provider of changes in the monitored implant such as a rupture 12.

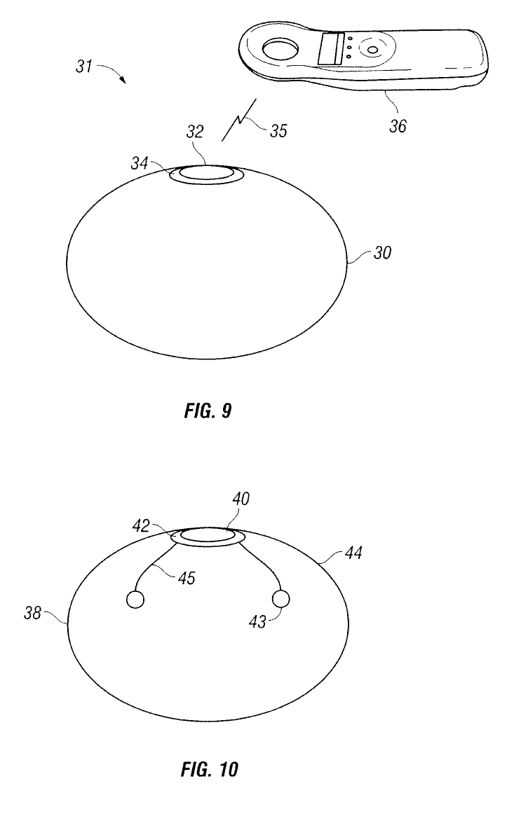

When implant 103 is so constructed that a bodily fluid 15 enters implant 103 upon a rupture 12 of shell 4 of implant 103, internal element 1 will emit a signal to power and/or signal emitter/receiver 16 to inform the user and/or healthcare provider that implant 103 has been compromised. A breach will be evident based on the change in the signal from a normal signal response 18 (FIGS. 7A and 7B) to that of a compromised implant signal response 19 (FIG. 7C). In response to the signal from the power and/or signal emitter/receiver 16, responsive signal 18, 19 may be generated by an active mechanism such an active RFID tag or other EMF, ultrasound or radiofrequency emitter, or may be generated by a passive mechanism such as a passive RPID tag. In other embodiments, a fluid contained in the implant will exit the implant upon a rupture of the implant shell and enter the surrounding environment, therefore, the system for external sensing of an implant rupture will be configured to detect the implant fluid exiting the implant. That is the case, for example, for breast implants filled with silicone gels. This embodiment will be described in greater detail with reference to FIG. 9.

Power and/or signal emitter/receiver 16 may be designed to interact intermittently with internal element 1 or may monitor the internal element 1 on a continuous basis. In some embodiments, power and/or signal emitter/receiver 16 may be placed within the home of the implant patient in an area that she will frequent at least once per day. For example, power and/or signal emitter/receiver 16 may be placed in, or near, a bed, chair, car, office, table or any other object or region that the implant patient will frequent on a daily basis. Moreover, power and/or signal emitter/receiver 16 may be powered by battery, capacitor, or from wall outlet and may be fixed in place or easily portable. Once powered up, power and/or signal emitter/receiver 16 will interact with internal element 1 and receive signals 18, 19 from internal element 1 to determine if the implant 103 has been compromised, as shown in FIG. 7C.

In the event that implant 103 is inflated within the body (for example, for breast implants and/or gastric balloons), implant 103 may be filled with an optional filling fluid 20 of known conductivity, capacitance, resistance and/or other electrical properties that vary significantly from normal saline and/or from bodily fluid 15 surrounding implant 103. Thus, by using internal element 1 to measure the electrical properties of filling fluid 20 and to detect variations in these properties upon mixing of filling fluid 20 with bodily fluids 15, a rupture 12 in the external shell 4 maybe sensed and communicated.

As previously discussed, in inflatable fluid- or gel-filled implants, an inflation patch is typically present somewhere on the implant. This inflation patch is typically formed from a much stronger material than the constituent materials of the implant shell and is added, usually by vulcanization, to the remainder of the implant shell after the shell has been fully manufactured.

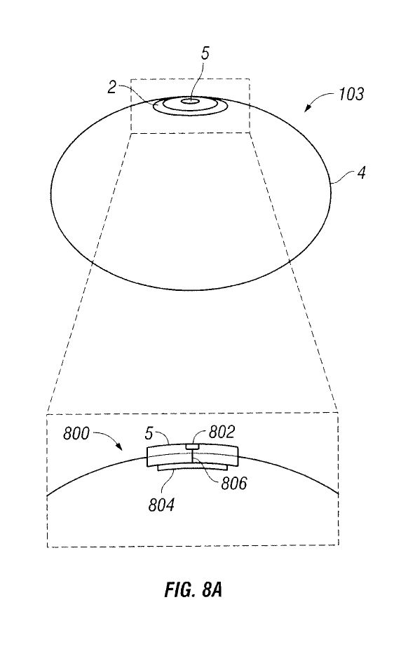

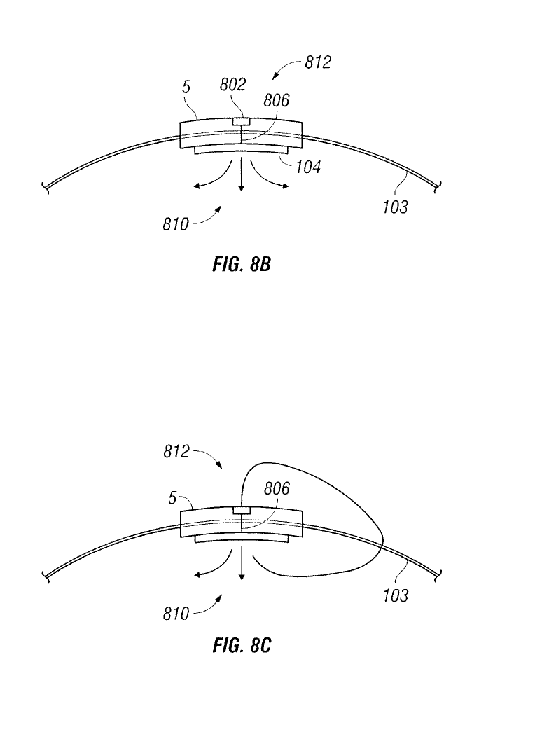

An external sensing system may alternatively include a sensor coupled with patch 5, whether implant 103 is filled with saline or another conductive fluid, or with a fluid having insulating properties such as a silicone gel. The embodiment of an external sensing system for an implant 103 filled with saline or other conductive fluid will be described first. In this embodiment, the sensor requires only a contact point on the inside of shell 4, which can be on patch 5 or free-floating with a connection to the patch 5, and an external contact point, which can simply be a small electrically conductive region on the outside of the implant. In this configuration, the only modification to implant 103 is required at patch 5 (and possibly within the filler) and no modification is required to shell 4, which is advantageous because any modification to shell 4 may increase the risk of rupture. This embodiment is depicted in FIGS. 8A-8C.

FIG. 8A is a perspective and enlarged view of injection patch 5 of an implant 103, which is filled with a conductive fluid or gel. The sensing and communicating components (for example, an RFID chip) of external sensing system 800 are incorporated within injection patch 5 as a chip 804, that is, shell 4 is unmodified. In the enlarged portion of FIG. 8A, an external electrical contact point 802 can be seen incorporated into a standard injection patch 5. This external electrical contact point 802 is in electrical communication with the electrical sensing and communicating chip 804 via electrical connection 806 spanning across patch 5.

As can be seen in FIG. 8B, in the presence of an intact shell 4, the electrical impulse released into conductive filling media 810 inside implant 103 by sensing and communicating chip 804 is exposed to an open circuit due to the insulating properties of the intact shell 4. As a result, none of the electrical impulse is transmitted to external electrical contact point 802.

In contrast, as can be seen in FIG. 8C, in the presence of a shell 4 that has a rupture, an electrical impulse released into conductive filling media 810 inside of the implant 103 by sensing and communicating chip 804 is in electrical communication with conductive bodily fluids 812 outside of the implant. As a result, an electric impulse is transmitted to external electrical contact point 802. As external electrical contact point 802 is in electrical communication with sensing and communicating chip 804 (via electrical connection 806 spanning patch 5), the now closed circuit allows the sensing and communicating chip 804 to receive an impulse from external electrical contact point 802 and, therefore, to report a rupture.

The patch-only modification found in FIGS. 8A-C may be used with the silicone gel embodiment by modifying the silicone gel to render it conductive (through the addition of metals, organometals, or other charge-carrying molecules to the silicone gel). Alternatively, the circumference of the silicone gel mass (at the gel-shell interface) may be made conductive while the central gel may be the standard, non-conductive gel. This may be accomplished through a two step gel insertion process whereby the outer rim of conductive gel is placed and cured (or partly cured) prior to installation and curing of the remainder of the non-conductive silicone gel. This approach will minimize the conductive silicone gel required and will provide a superior solution compared to conductive layers or meshes within the shell in that the silicone gel emanating from the tear will not coat and insulate the conductive layer if it is the conductive layer itself. In addition to the standard dip-molding of the shell and injection of the silicone gel, the layered and/or conductive silicone gel approach could also be manufactured using single or multiple shot molding processes. In this embodiment, the device may or may not be radiolucent.

While the embodiment shown in FIGS. 8A-8C is shown as being used with a silicone device with a shell 4 and conductive filling media 810, the implant integrity monitoring device 800 could also be used with any implant 103 that has a non-conductive shell 4. For example, in the instance of a pacemaker or implantable cardioverter defibrillator, device 800 could be used in the shell of the implant near the most likely point of fluid ingress. The device 800 may then be interrogated routinely to determine if the shell has been compromised via the detection of the ingress of conductive bodily fluids. Further, while the embodiment shown in FIGS. 8A-8C has been described as being fully incorporated into the patch of the implant, some element of device 800 may be included within the implant or within the external milieu (e.g., in the manner of the tethers of embodiments shown in FIGS. 1-4), so long as an external communication exists across implant shell 4. Finally, whereas the embodiment shown in FIGS. 8A-8C is described as monitoring an internal conductivity of fluid 810 within implant 103, other embodiments of the present invention envision simultaneously monitoring both fluid 810 within implant 103 and the fluid 812 outside of the implant 103 to determine the presence or absence of a complete conducting pathway across shell 4 of the implant 103.

When an implant such as a breast implant is filled with a silicone gel, both shell 4 and the silicone gels are non-conductive electrically and, in addition, tracking the ingress of bodily fluids into an implant filled with silicone gels is problematic, because the bodily fluids tend not to enter implant 103 through a rupture in shell 4 but, on the contrary, the silicone gel tends to exit implant 103. As a result, the external sensing of a rupture for an implant 103 that has a non-conductive filling through sensing systems disclosed in the prior art would likely require modifications to the entire shell to sense the outflow of silicone from the implant. Such a configuration would be time-consuming and costly to implement, and would add a future risk of failure (for example, from perforation or rupture) due to the required modifications of shell 4.

FIG. 9 illustrates an embodiment of an external sensing system 31 for detecting a rupture in a silicone gel implant, in which a sensor 32 is coupled to patch 34 of implant 30. In this embodiment, sensor 32 includes circuitry, a radio-frequency identification (“RFID”) element, an antenna and, optionally, a power source that are all incorporated within durable patch 32 of device 30. The patch at the back of implant 30 and may include an optional external opening and closing of the antenna element to allow for better MRI compatibility. Sensor 32 includes electrical contacts that are electrically coupled when implant 30 is intact but that become coated with a non-conductive material such as a silicone gel and electrically decoupled in the event of a rupture in implant 30. Those electrical contacts may interrogated by the RFID chip once it receives a powering signal from an external transmitter 36, and a lack of conduction between those electrical contacts is indicative of the presence of insulating silicone gel.

In a variant of this embodiment, the RFID chip may employ a capacitor or other temporary power storage device capable of storing energy received from external transmitter 36 until a threshold is reached, at which point sensor 32 is interrogated and a signal may be released from sensor 32 to external transmitter 36. In another variant, a powerful signal may be released for longer-range communication with a remote external transmitter/sensing device. The RFID chip, or other communicating element, may also incorporate other functionality, such as identification of the implant for tracking and maintenance purposes.

FIG. 10 illustrates a second embodiment of an external sensing system for a silicone gel implant 38, in which a sensor 40 is coupled to a patch 42, as well as to shell 44 and/or the lumen of implant 38 by adding one or more additional sensors 43 to shell 44 and/or to the lumen of the implant. Sensor 40 and additional sensors 43 are electrically coupled with leads 45. This embodiment allows for redundant interrogation of the implant and earlier detection of rupture in the event that the silicone gel is tracking slowly. At the same time, this embodiment requires a modification of shell 44 and/or the incorporation of additional hardware into implant 38. A new line of silicone breast implants includes areas of increased thickness on contoured implants and these regions of increase thickness could be used for silicone doping with electrically active material and/or incorporation of conductive electrodes. In a variant of this embodiment, no sensor 4 is coupled to patch 42, but only one or more sensors 43 are disposed on shell 44 and/or in the lumen of implant 38.

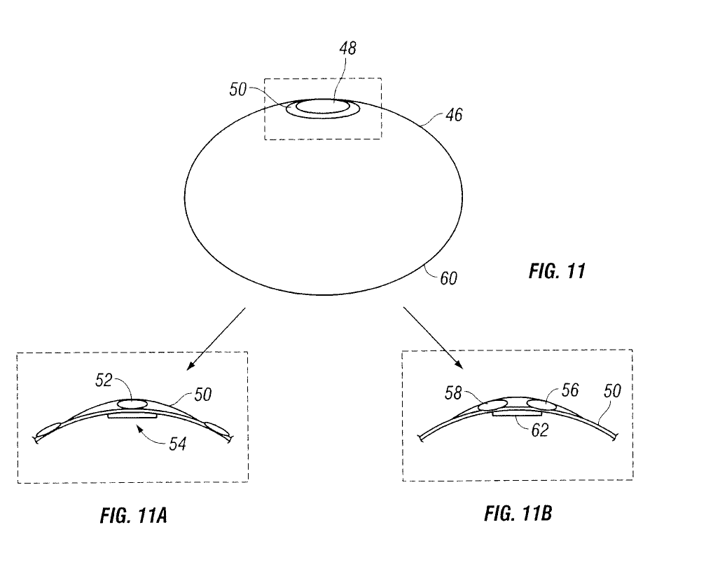

FIGS. 11, 11A and 11B illustrate the structure of an embodiment of the present invention for a silicone gel implant 46. In this embodiment, schematically illustrated in FIG. 11, a sensor 48 is coupled to patch 50. Sensor 48 is configured to interrogate the external milieu for the presence or absence of implant contents, more specifically, of silicone gel. More particularly, FIG. 11A illustrates the configuration of a sensor 52 that includes one or more elements capable of detecting visual, pH, chemical, acoustic, electrical, viscosity, spectrophotometric changes or changes in other properties caused by an exit of a filler from breast implant 46. For example, sensor 52 may detect chemical changes or photo-distortion of an image. A circuit or chip 54 may be disposed either within patch 50 or, as shown in FIG. 11A, on the inside of patch 50.

FIG. 11B illustrates instead a sensor 56 that includes one or more elements 58 (for example, electrodes) that detect changes in the conductivity or other electrical properties in the surrounding pocket due to a coating of element or elements 58 by the implant filler. If the implant filler has insulating properties, an otherwise closed circuit between elements 58 becomes open to the coating by the implant filler of one or more of elements 58.

Elements 58 may protrude from sensor 56 at different heights. In a preferred embodiment, illustrated in FIG. 11B, elements 58 are flush with (that is, do not protrude from) the outer surface of patch 50 to facilitate coating of patch 50 with the breast implant filler in the event of a breach in shell 62. In one variant of the present embodiment, sensor 58 is inset into shell 60 of implant 46. Even in this embodiment, a circuitry or chip 62 is disposed or printed on the wall of patch 62 facing shell 46. In this variant, it would be preferable to employ an implant 46 having a shell 60 of varying thickness, so that sensor 56 can be inset in a thicker portion of shell 60 and a breach point in shell 60 is not induced at the pocket housing sensor 58.

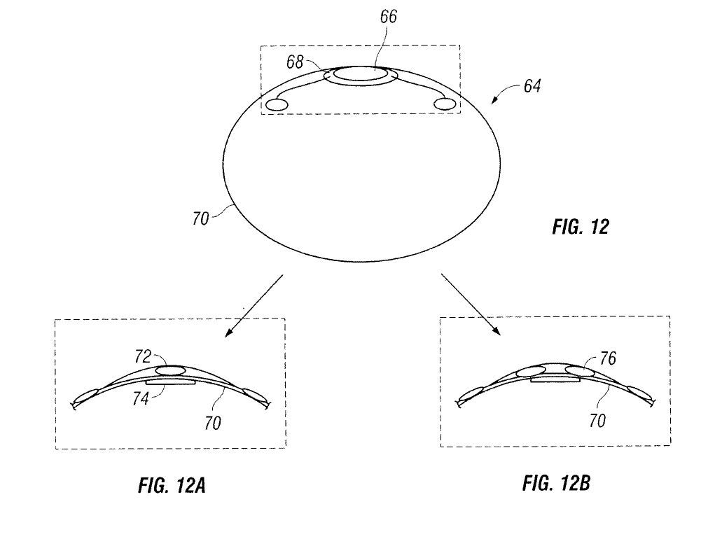

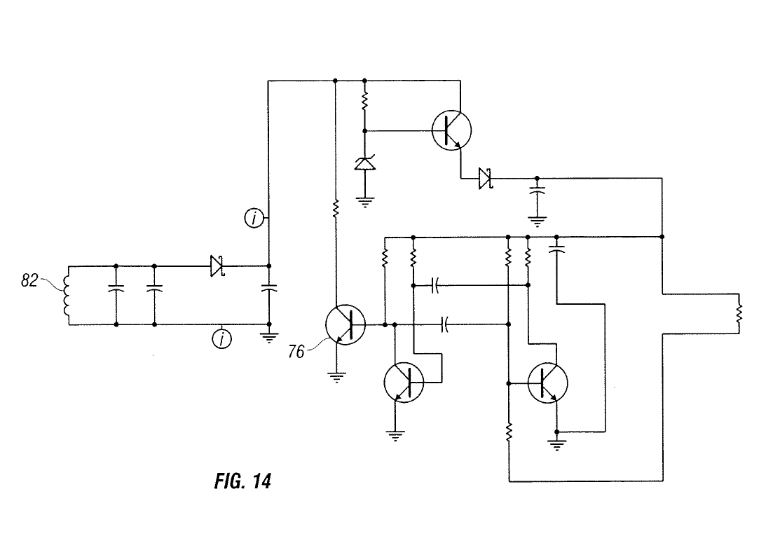

Referring now to FIG. 12, another embodiment of the invention relates to an external sensing system for a silicone gel implant 64, in which a sensor 66 is incorporated within the implant patch 68 as well as the shell 70 and/or lumen of implant 64. Sensors 66, incorporated within patch 68, shell 70 or the lumen of implant 64 is capable of interrogating the external milieu for the presence or absence of implant contents, in particular, of silicone gel.

More particularly, FIG. 12A illustrates that one or more sensors 72 may be incorporated within a reinforced area of shell 70 and may detect visual, pH, chemical, acoustic, electrical, viscosity, spectrophotometric, or other properties associated with an exit of a filler from breast implant 64. For example, sensors 72 may be one or more chemical or photo sensors configured to detect chemical changes or photodistortion of an image in the external pocket. A circuit or chip 74 may be disposed within shell 70 and be coupled to sensors 72 to receive energy from an external reader, as described in greater detail below, activate sensors 72 and/or process signals or data provided by sensors 72.

Referring now to FIG. 12B, in a different embodiment one or more sensors 76 may be provided as contacts (for example, electrodes) on the outside of the shell 70 and these electrical contacts may be electrically connected one to the other, forming a closed circuit. A leak of silicone gel from implant 64 will hinder or prevent conduction among the electrical elements by insulating one or more of the electrical elements, thereby opening the circuit. Therefore, upon interrogation by an external reader, no conductivity will be detected among the contacts, indicating a likely silicone leak that may be confirmed through a MRI scan. Multiple redundant checks may be planned for added sensitivity and precision.

The previous description has outlined the basic components of the embodiments of the invention related to sensing an insulating filler (such as a silicone gel) upon a leak from an implant. Those components will be described in greater detail hereinafter.

A system 31 for external sensing for implant rupture configured as in the embodiments depicted in FIG. 9 includes two basic components, a sensor 32 that operates as a receiver and transmitter, and an external reader and/or transmitter wand 36. Sensor 32 may be a RFID chip that is firmly bonded to the strongest portion of implant 30, patch 34, and that may then be queried, post-implantation, to determine the conductivity of the capsular milieu. The basic premise behind this embodiment is that the conductivity will be stable in the presence of saline (the normal capsular fluid), but will dramatically decrease in the presence of capsular silicone.

Sensor 32 includes a silicone-encapsulated electronic circuit and is attached to the posterior surface of a silicone breast prosthesis 30, where it detects leakage of silicone gel and transmits this information to a transmitter wand 36, when queried, via a wireless radio-frequency link 35. In the preferred embodiment, sensor 32 is affixed to patch 34 through a process that will provide a reasonable expectation that sensor 32 will not become decoupled from patch 34 during the life if implant 30, for example, by bonding with an adhesive that will not decompose under the influence of bodily fluids or by vulcanization.

Referring now again to FIGS. 11 and 11B, leakage of silicone gel is sensed as a change in conductivity between two or more electrodes 58 on the surface of implant 46 or, as shown, on the surface of patch 50. Normally there is conduction between the electrodes due to the salinity of bodily fluid and tissues. If there is leakage of the silicone gel, the gel will coat electrodes 58 with a non-conductive film. Benchtop studies have confirmed that silicone gel will rapidly track from the apex of a silicone gel implant to the area of sensor 48 with physiologic agitation. These tests were conducted using the latest generation, most highly cohesive silicone gel implants from Mentor and Allergan.

Transmitter wand 36 is easy to use and provides inductive power to the sensor 32 with the push of a button in a completely noninvasive manner. Once powered, the electronic circuit of sensor 32 senses the resistance between electrodes 58 and encodes it as a pulsed waveform, with the pulse frequency related to the resistance. The coded information is transmitted via a radio signal at 13.56 MHz to a coil 37 in transmitter wand 36.

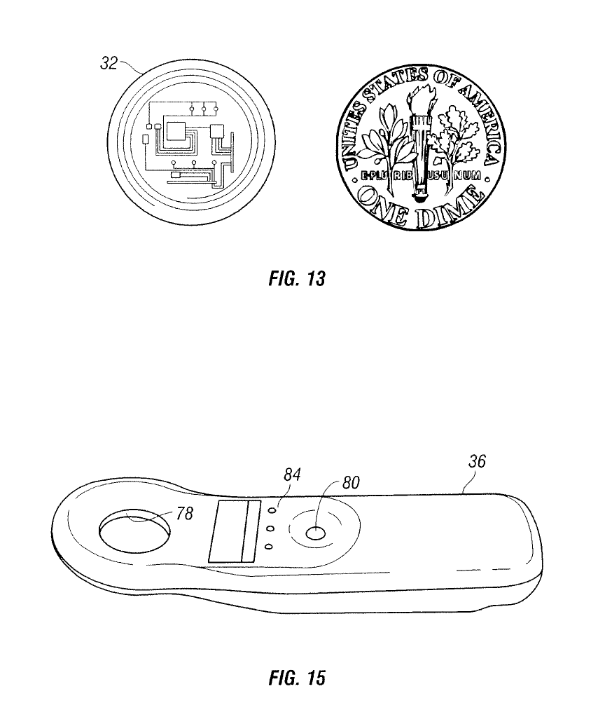

An embodiment of sensor 32 is illustrated in FIG. 13, which shows, among other things, the relative size of sensor 32 in comparison to a U.S. ten cent coin. Sensor 32 has a tuned coil to receive RF energy from transmitter wand 36, which emits a signal that is rectified, filtered, and regulated to provide direct current (“DC”) power.

A portion of the circuitry of sensor 32 is depicted in detail in FIG. 14, which shows that the oscillator consists of an astable multivibrator using two transistors 76. A crystal-controlled oscillator produces an output that is amplified by a transistor, and the amplifier output is coupled to a resonant coil through a matching network. The voltage on the coil is monitored by a diode detector, the output of which is connected to a comparator. When the comparator senses a signal greater than a set threshold, the comparator produces a pulse output, which is sensed by a small microprocessor measuring the pulse period. The pulse period is related to the measured resistance, and the microprocessor determines whether the resistance is within selected limits, sending that data to transmitter wand 36.

Sensor 32 is fully encapsulated within silicone, with the exception of the platinum-iridium electrodes that are flush with, but exposed at, the surface of silicone coating. The construction of sensor 32 is such that sensor 32 can be easily incorporated within, or placed on the outside of, the patch of the breast implant. In benchtop models using a simulated tissue capsule and gentle agitation, sensor 32 was found capable of detecting capsular gel placed anywhere on implant 30. Even in the worst-case scenario of cohesive silicone gel presentation at the apex of implant 30, mild agitation was found to result in grossly visible distribution of the gel over the entire surface of implant 30.

Referring now to FIG. 15, transmitter wand 36 consists of a hand-held transmitter producing about one Watt of RF output to a coil 78 when a power button 80 is depressed. The RF energy is coupled to a coil 82 in implant 30 and supplies power to sensor 32, which includes a multivibrator oscillator having a frequency determined by the resistance between external sensing electrodes (for example, electrodes 58 of FIG. 11B). Sensor 32 modulates the signal received from transmitter wand 36, and this modulation is reflected back to transmitter coil 78 and can be detected to reconstruct the frequency of the oscillator of sensor 32. This communication is similar to the coupling between the windings of a transformer, where the load on the secondary winding is reflected in changes in the primary winding.

LED indicators 84 on wand 36 indicate the conductivity between the electrodes of sensor 32. For example, when a LED indicator 84 is green, the conductivity between the electrodes in sensor 32 is within the normal range, but when a LED indicator on the Wand is red, conductivity is abnormally low, indicating the presence of capsular silicone.

A system according to the present invention may be utilized with breast implants 30 that are round, contoured or of other shape, that are symmetric or asymmetric, or with other asymmetric implant designs.

The description of one method of use of a system according to the present invention follows. A person skilled in the art will appreciate that, while a method of use is described with reference to a breast implant filled with silicone gel, this method is equally applicable to other types of implants. A person skilled in the art will further appreciate that methods having different but equivalent steps may also be employed and fall within the scope of the present invention.

In a first step, described with reference to FIG. 9, a sensor 32 is disposed on the outer surface of a breast implant 30. Preferably, sensor 32 is coupled to patch 34 by a process that will insure that sensor 32 does not become accidentally decoupled from patch 34 during the life of the implant, for example, by vulcanization or by adhesive bonding with an adhesive that is impervious to bodily fluids and contents. Alternatively, sensor 32 may be disposed within a recess on the shell of implant 30, preferably in a portion of the shell with increased thickness in comparison to the rest of the shell.

In a second step, implant 30 is inserted in the body of a patient, for example with a surgical procedure. Sensor 32 will be disposed within the patient's body in a position that provides adequate reception from and transmission to a reader outside of the patient's body and at the same time is suitable for the intended use of the implant, for example, sensor 32 will be disposed towards the inner part of the body, so that it cannot be sensed during a palpation of the breast.

In a third step, a transmitter/received device is provided, for example, the transmitter/receiver wand 36 described with reference to FIG. 15.

In a fourth step, wand 36 is positioned outside of the patient's body in the proximity of implant 30, for example, in front of the breast containing implant 30. This step may be performed by a healthcare provider, for example, at the time of a mammography, or may be performed by the patient herself.

In a fifth step, energy is provided telemetrically from wand 36 to sensor 32 by depressing button 80 (FIG. 15), energizing sensor 32 and causing sensor 32 to read the level of electrical conduction between electrodes 58 (FIG. 11B). If sensor 32 read that conduction is within a predetermined range, a green LED indicator 84 will be lit, indicating that the sensor has not detected insulators between electrodes 58 and providing an indication that no silicone gel has exited implant 30. On the contrary, if sensor 32 reads a conduction level below a predetermined level (for example, that conduction is non-existent), a red LED indicator 84 will be lit, indicating that an insulator between electrodes 58 has been detected and that silicone gel may have exited implant 30. In that event, the patient or healthcare professional would likely schedule a MRI scan to confirm the reading of wand 15. A third LED indicator 84 may also be lit if a malfunction is in the operation of the external sensing system is detected, for example, that sensor 32 is not receiving energy from wand 36.

The present invention has been envisioned as being highly useful for any inflatable implant, including breast implants, percutaneous gastrostomy tubes, Foley catheters, penile implants, gastric balloons, etc. Further, due to the relative ease of measuring electrical properties, the sensor could be reduced significantly in size or even simply encompass an RFID and electrical property sensing element that are printed in a suitable location of the implant to be monitored, for example, on patch 34 of FIG. 9. In this way, changes in electrical properties can be quickly and easily measured and reported in a very low-profile manner within or outside of the implant. This feature may also apply to other characteristics of the filling fluid including chemical, optical, physical, pH, electrical properties, etc.

Lastly, while RFID has been mentioned as a communicating mechanism, a variety of other mechanisms may be employed including auditory, acoustic, vibrational or other stimuli to alert the patient that the implant has been compromised. In addition, while RFID has also been mentioned as a method of powering the device, the device may also be powered by alternative mechanisms, including a self-winding mechanism (as found in watches), an internal rechargeable battery, or a long-lasting capacitor/internal battery. These alternative charging and alerting mechanisms all provide for an additional safeguard in that the patient may be notified nearly instantaneously of a rupture and not require the additional step of exposure to an RFID transmitting/receiving apparatus.

While the invention has been described in connection with the above described embodiments, it is not intended to limit the scope of the invention to the particular forms set forth, but on the contrary, it is intended to cover such alternatives, modifications, and equivalents as may be included within the scope of the invention. Further, the scope of the present invention fully encompasses other embodiments that may become obvious to those skilled in the art and the scope of the present invention is limited only by the appended claims.

What is claimed is:

1. A system for detecting rupture of an implant in a body, the system comprising:
a sensor disposed on a surface of the implant, the sensor being configured to detect a property of a surrounding environment and to emit a wireless signal, the property being detected entirely outside or entirely inside the implant and indicating whether the rupture has occurred; and
a device external to the body and configured to receive the wireless signal from the sensor.

2. The system of claim 1, wherein the sensor is printed on the surface of the implant.

3. The system of claim 1, wherein the sensor is coupled to a patch coupled to the implant.

4. The system of claim 1, wherein the sensor is disposed in a recess provided in a reinforced area of the surface.

5. The system of claim 1, wherein the surface is an outer surface or an inner surface.

6. The system of claim 1, wherein the sensor comprises a plurality of electrodes coupled to the surface of the implant, and wherein the property is electrical conduction between the electrodes.

7. The system of claim 6, wherein the sensor comprises a multi-vibrator oscillator having a frequency determined by a resistance between the electrodes.

8. The system of claim 6, wherein the electrodes are arranged on the surface to provide a profile flush with the surface.

9. The system of claim 6, wherein the implant comprises a filler having insulating properties, wherein the surface is an outer surface, and wherein the sensor is configured to measure a reduction in electrical conduction between the electrodes after the rupture.

10. The system of claim 1, wherein the sensor comprises a radio-frequency identification circuit.

11. The system of claim 1, wherein the signal comprises data.

12. The system of claim 1, wherein the system transmits a radio signal at a frequency of about 13.56 MHz.

13. The system of claim 1, wherein the sensor is configured to receive power transmitted from the device.

14. The system of claim 14, wherein the sensor is configured to receive power from the device inductively at about one Watt of radio-frequency output.

15. The system of claim 1, wherein the sensor comprises an oscillator, and wherein the oscillator comprises an astable multivibrator.

16. The system of claim 1, wherein the sensor comprises a microprocessor detecting a change in the property by comparing a reading of the property against a predetermined threshold.

17. The system of claim 1, wherein the device comprises a display of whether the signal indicates that the implant rupture has occurred.

18. A method for detecting rupture of an implant in a body, the method comprising:
disposing a sensor on a surface of the implant, the sensor being configured to detect a property of a surrounding environment and to emit a wireless signal, the property being detected entirely outside or entirely inside the implant and indicating whether the rupture has occurred;
providing a device external to the body and configured to receive the signal from the sensor wirelessly;
connecting the device with the sensor wirelessly; and
receiving an alert from the device in the event the implant rupture has occurred.

19. The method of claim 18, wherein coupling the sensor to the surface comprises coupling the sensor to a patch coupled to the implant.

20. The method of claim 18, wherein coupling the sensor comprises providing the sensor with a radio-frequency identification circuit.

21. The method of claim 18, wherein coupling the sensor comprises coupling a plurality of electrodes to an inner or outer surface of the implant, and wherein the property is electrical conduction between the electrodes.

22. The method of claim 21, wherein coupling the plurality of electrodes to the outer surface of the implant comprises coupling the plurality of the electrodes to provide a profile flush with the outer surface.

23. The method of claim 21, wherein coupling the sensor comprises coupling a plurality of electrodes to the outer surface of the implant, wherein receiving the alert comprises receiving a signal that electrical conduction between the electrodes has decreased due to an insulating implant filler contacting one or more of the electrodes.

24. The method of claim 18, further comprising the step of providing power from the device to the sensor wirelessly.

25. The method of claim 18, further comprising the step of having the device provide a display of whether the signal indicates that the implant rupture has occurred.
